(19) United States
(12) Patent Application Publication
(10) Pub. No.: US 2021/0358615 A1
(43) Pub. Date: Nov. 18, 2021

(54) MAINTENANCE SYSTEM, MAINTENANCE METHOD, AND PROGRAM

(71) Applicant: TOYOTA JIDOSHA KABUSHIKI KAISHA, Toyota-shi (JP)

(72) Inventor: Norihiko KAWADA, Toyota-shi (JP)

(73) Assignee: TOYOTA JIDOSHA KABUSHIKI KAISHA, Toyota-shi (JP)

(21) Appl. No.: 17/221,385

(22) Filed: Apr. 2, 2021

(30) Foreign Application Priority Data
May 15, 2020 (JP) 2020-086050

(51) Int. Cl. G16H 40/67; A61H 3/00; A61B 5/00; A61B 5/103; G16H 20/30

(52) U.S. Cl. CPC G16H 40/67; A61H 3/00; A61B 5/6807; A63B 22/02; A61B 5/4836; G16H 20/30; A61B 5/1038

(57) ABSTRACT

A maintenance system includes: a walking training apparatus including: a walking assistance apparatus including a foot attaching part attached to a foot of a trainee, and load detection means for detecting a load occurring on a sole of the trainee in the foot attaching part, the walking assistance apparatus being configured to assist the trainee in walking; and first transmission means for transmitting information about the detected load and information about a time during which the load is exerted; and a server including: determination means for comparing the information about the load and the information about the time during which the load is exerted with a preset evaluation value for durability of the walking assistance apparatus, and determining a timing for performing maintenance for the walking assistance apparatus; and second transmission means for transmitting the determined timing of the maintenance as maintenance information.

### CROSS REFERENCE TO RELATED APPLICATIONS

This application is based upon and claims the benefit of priority from Japanese patent application No. 2020-086050, filed on May 15, 2020, the disclosure of which is incorporated herein in its entirety by reference.

### BACKGROUND

The present disclosure relates to a maintenance system, a maintenance method, and a program.

A walking training apparatus including a walking assistance apparatus which is attached to a leg of a user and assists the user in walking has been known (see, for example, Japanese Patent No. 6554996).

### SUMMARY

As the user performs walking training, the walking assistance apparatus repeatedly receives loads (i.e., pressures) from the leg of the user because of the walking. Because of these loads caused by the walking, each of the components constituting the walking assistance apparatus is worn and eventually requires maintenance. Therefore, it has been desired to develop a maintenance system capable of providing an appropriate maintenance timing that is not too early and not too late.

The present disclosure has been made to solve the above-described problem and an object thereof is to provide a maintenance system, a maintenance method, and a program capable of providing an appropriate maintenance timing.

A first exemplary aspect is a maintenance system including:

a walking training apparatus including:

a walking assistance apparatus including a foot attaching part configured to be attached to a foot of a trainee, and load detection means for detecting a load occurring on a sole of the trainee, the load detection means being disposed in the foot attaching part, and the walking assistance apparatus being configured to assist the trainee in walking; and

first transmission means for transmitting information about the load detected by the load detection means and information about a time during which the load is exerted; and

a server including:

determination means for comparing the information about the load and the information about the time during which the load is exerted, which have been transmitted from the first transmission means, with a preset evaluation value for durability of the walking assistance apparatus, and determining a timing for performing maintenance for the walking assistance apparatus; and

second transmission means for transmitting the timing of the maintenance determined by the determination means as maintenance information.

In this aspect, the first transmission means may transmit the load detected by the load detection means and the load time during which the load is exerted to the server, and the determination means of the server may calculate an integral value obtained by integrating the load transmitted from the first transmission means over the load time, compare the calculated integral value with the evaluation value for the durability, and determine the timing for performing the maintenance for the walking assistance apparatus.

In this aspect, the first transmission means may transmit, to the server, information about at least one of a skeletal movement of the trainee, a center of a load exerted on the sole, and a knee joint angle detected by a sensor provided in the walking training apparatus together with the information about the load and the information about the time during which the load is exerted; the server may further include bias determination means for determining a direction of a bias of the load in the walking of the trainee based on the information about at least one of the skeletal movement of the trainee, the center of the load exerted on the sole, and the knee joint angle transmitted from the first transmission means; the evaluation value for the durability may be set according to the direction of the bias of the load; and the determination means may set the evaluation value for the durability according to the direction of the bias of the load in the walking of the trainee determined by the bias determination means, compare the set evaluation value for the durability with the information about the load and the information about the time during which the load is exerted, which have been transmitted from the first transmission means, and determine the timing for performing the maintenance for the walking assistance apparatus.

Another exemplary aspect may be a maintenance method including:

detecting a load occurring on a sole of a trainee to which a walking assistance apparatus is attached, and the walking assistance apparatus being configured to assist the trainee in walking;

transmitting, to a server, information about the detected load and information about a time during which the load is exerted;

comparing, by the server, the transmitted information about the load and the information about the time during the load is exerted with a preset evaluation value for the durability of the walking assistance apparatus, and determining a timing for performing maintenance for the walking assistance apparatus; and

transmitting, by the server, the determined timing of the maintenance as maintenance information.

Another exemplary aspect may be a program for causing a computer to perform:

a process for detecting a load occurring on a sole of a trainee to which a walking assistance apparatus is attached, and the walking assistance apparatus being configured to assist the trainee in walking;

a process for transmitting, to a server, information about the detected load and information about a time during which the load is exerted;

a process for comparing, by the server, the transmitted information about the load and the information about the time during the load is exerted with a preset evaluation value for the durability of the walking assistance apparatus, and determining a timing for performing maintenance for the walking assistance apparatus; and

a process for transmitting, by the server, the determined timing of the maintenance as maintenance information.

According to the present disclosure, it is possible to provide a maintenance system, a maintenance method, and a program capable of providing an appropriate maintenance timing.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing a schematic system configuration of a maintenance system according to an embodiment;

FIG. 2 is a perspective view showing a schematic configuration of a walking training apparatus according to an embodiment;

FIG. 3 is a side view showing a schematic configuration of a walking assistance apparatus according to an embodiment;

FIG. 4 is a perspective view showing a schematic configuration of a sole frame according to an embodiment;

FIG. 5 is a block diagram showing a specific system configuration of a maintenance system according to an embodiment;

FIG. 6 is a block diagram showing a schematic system configuration of an information providing server according to an embodiment;

FIG. 7 is a flowchart showing a flow of a maintenance method according to an embodiment; and

FIG. 8 is a block diagram showing a schematic system configuration of an information providing server according to an embodiment.

### DESCRIPTION OF EMBODIMENTS

### First Embodiment

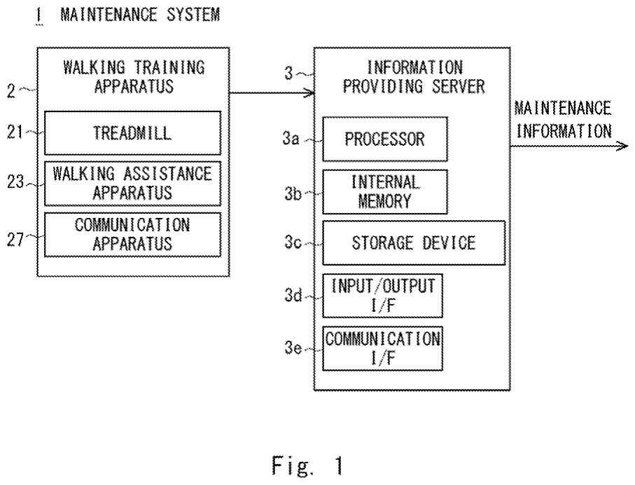

Embodiments according to the present disclosure will be described hereinafter with reference to the drawings. FIG. 1 is a block diagram showing a schematic system configuration of a maintenance system according to an embodiment. A maintenance system 1 according to this embodiment includes a walking training apparatus 2 by which a trainee performs walking training, and an information providing server 3 which provides maintenance information.

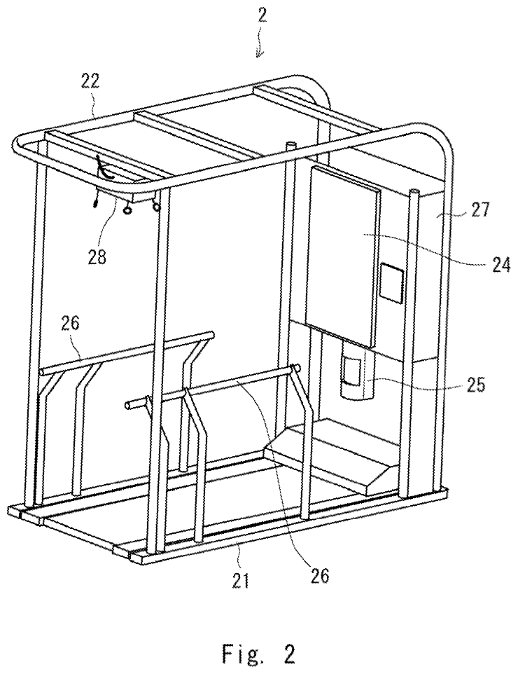

FIG. 2 is a perspective view showing a schematic configuration of the walking training apparatus according to this embodiment. The walking training apparatus 2 is provided in a hospital or a rehabilitation facility. The walking training apparatus 2 includes a treadmill 21, a frame main body 22, a walking assistance apparatus 23, and the like. The treadmill 21 rotates a ring-shaped belt. A trainee performs walking training by getting on the belt and performing walking according to the movement of the belt.

The frame main body 22 is composed of a plurality of frames connected above the treadmill 21. In the frame main body 22, a pulling machine 28 that pulls the trainee upward and thereby assists him/her, a monitor 24 that displays various information items, a sensor(s) 25 that detects a walking state of the trainee, handrails 26 that the trainee grasps during the training, a communication apparatus 27 that transmits/receives data, and the like are provided.

The monitor 24 displays information about, for example, training instructions, a training menu, and training information (such as a walking speed and biological information). The sensor 25 is, for example, a 3D (three dimensional) sensor capable of detecting the skeletal movement of the trainee. The 3D sensor acquires a depth image showing projections and depressions on the body surface of the person (e.g., the trainee) by irradiating the body surface with laser light, and recognizes the skeletal structure of the person by extracting 3D coordinates of joints from the depth image. The sensor 25 transmits the detected skeletal movement of the trainee to the communication apparatus 27.

The communication apparatus 27 is a specific example of the first transmission means. The communication apparatus 27 is connected to the walking assistance apparatus 23 through wireless communication such as Bluetooth (Registered Trademark) or Wifi (Registered Trademark), or through wired communication. The communication apparatus 27 can transmit/receive data to/from the walking assistance apparatus 23. Further, the communication apparatus 27 can transmit/receive data to/from the information providing server 3 through a communication network such as the Internet.

Note that the communication apparatus 27 may be disposed in the walking assistance apparatus 23. The communication apparatus 27 transmits information about the walking assistance apparatus 23 received from the walking assistance apparatus 23. The walking training apparatus 2 may not be necessarily equipped with the treadmill 21 and the frame main body 22.

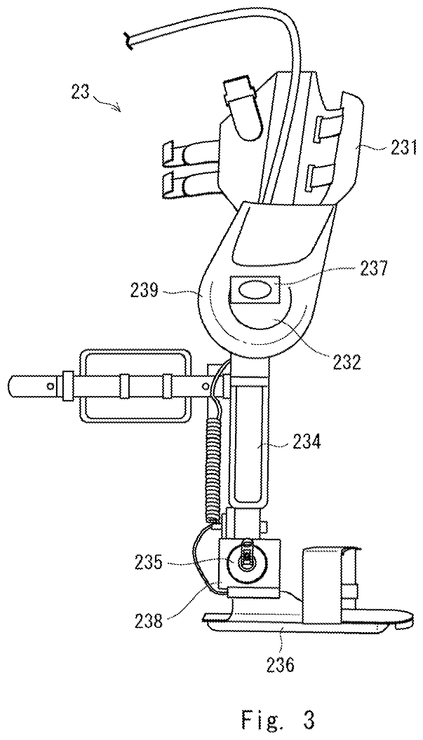

FIG. 3 is a side view showing a schematic configuration of the walking assistance apparatus according to this embodiment. The walking assistance apparatus 23 is attached to a leg of a trainee and assists the trainee in walking. The walking assistance apparatus 23 includes a thigh frame 231, a lower-leg frame 234 connected to the thigh frame 231 through a knee joint part 232, a sole frame (a specific example of the foot attaching part) 236 connected to the lower-leg frame 234 through an ankle joint part 235, a motor unit 237 that rotationally drive the knee joint part 232, and an adjustment mechanism 238 that adjusts the movable range of the ankle joint part 235.

An angle sensor 239 that detects the angle of the knee joint part 232 is provided in the knee joint part 232. Note that the above-described configuration of the walking assistance apparatus 23 is merely an example, and its configuration is not limited to this example. For example, the walking assistance apparatus 23 may include a motor unit that rotationally drives the ankle joint part 235.

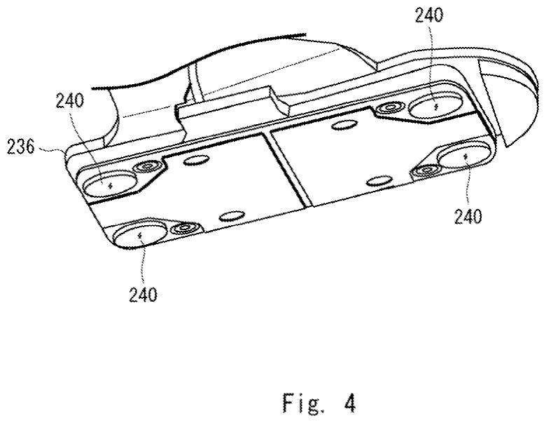

FIG. 4 is a perspective view showing a schematic configuration of the sole frame. In the sole frame 236, a pair of load sensors 240 is provided on each of the toe side and the heel side on the sole surface. The load sensors 240 are a specific example of the load detection means. Each of the load sensors 240 is, for example, a vertical load sensor that detects a load (i.e., a pressure) exerted in a direction perpendicular to the sole of the sole frame 236. Note that the number and positions of the load sensors 240 provided in the sole frame 236 may be arbitrarily determined.

The walking assistance apparatus 23 calculates the position of the center of the load (COP: Center of Pressure) exerted on the sole (hereinafter referred to as the COP position) during the gait motion performed by the trainee based on the load detected by the load sensors 240.

For example, the center position on the sole of the sole frame 236 is defined as the origin of a 2D (two dimensional) XY-coordinate system. Then, the position of a first load sensor 240 on the toe side is expressed as (x1, y1), and the position of a second load sensor 240 on the toe side is expressed as (x2, y2). Further, the position of a third load sensor 240 on the heel side is expressed as (x3, y3), and the position of a fourth load sensor 240 on the heel side is expressed as (x4, y4). A load value exerted on each of the load sensors 240 is represented by Ni (i=1 to 4). The walking assistance apparatus 23 calculates the COP position (x_{COP}, y_{COP}) by using, for example, the below-shown expressions.

$${x}_{COP} = \frac{{\sum }_{i = 1}^{4} {N}_{i} {x}_{i}}{{\sum }_{i = 1}^{4} {N}_{i}} \\ {y}_{COP} = \frac{{\sum }_{i = 1}^{4} {N}_{i} {y}_{i}}{{\sum }_{i = 1}^{4} {N}_{i}} \qquad [ Expression 1 ]$$

The above-described method for calculating a COP position is merely an example, and the method is not limited to this example. For example, instead of using the load sensors 240, a load distribution sensor that detects a distribution of loads (i.e., pressures) on the sole may be provided on the sole of the sole frame 236. The walking assistance apparatus 23 may calculate the COP position based on the distribution of loads on the sole detected by the load distribution sensor.

The walking assistance apparatus 23 transmits, to the communication apparatus 27, various information items such as load values detected by the load sensors 240, a load time (i.e., a load duration) during which the load is exerted, the position of the COP, and the angle of the knee joint detected by the angle sensor 239. The load values are an example of the information about the load. The load time (i.e., the load duration) is an example of the information about the time during which the load is exerted.

It should be noted that as a user performs walking training, the walking assistance apparatus 23 repeatedly receives loads (i.e., pressures) from the leg of the user because of the walking. Because of these loads caused by the walking, each of the components constituting the walking assistance apparatus 23 is worn and eventually requires maintenance.

For example, due to the loads caused by the walking, each of the components of the walking assistance apparatus 23 may slightly deform, creep, and/or be worn, so that the values obtained by the load sensors 240 of the sole frame 236 may deviate (i.e., change) from their initial values. In such a case, it is necessary to calibrate the load sensors 240.

In the case in which the timing of such maintenance is set, if the maintenance period is set to a long span, the load sensors are left unattended in the above-described inaccurate state for a certain period. On the other hand, if the maintenance period is set to a short span, the walking assistance apparatus is frequently stopped for the maintenance, so that the efficiency of the rehabilitation operation is lowered.

Therefore, it has been desired to develop a maintenance system capable of providing an appropriate maintenance timing that is not too early and not too late. To this end, the maintenance system 1 according to this embodiment includes the information providing server 3 that provides maintenance information including a timing for performing maintenance for the walking assistance apparatus 23 to a maintenance company or the like.

As a result, the maintenance company or the like can perform maintenance for the walking assistance apparatus 23 at an appropriate maintenance timing by using the maintenance information provided from the information providing server 3.

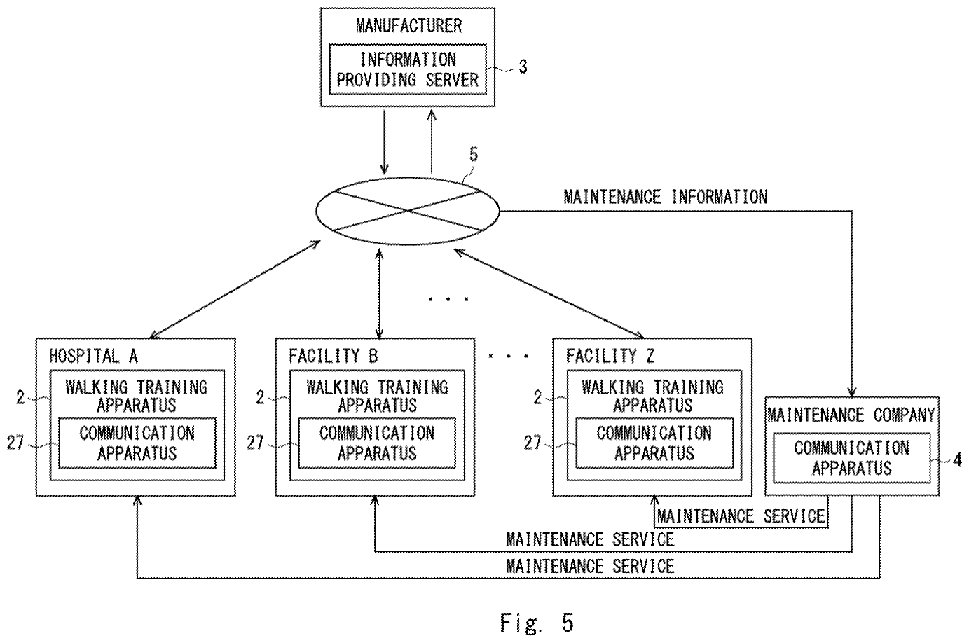

FIG. 5 is a block diagram showing a specific system configuration of the maintenance system according to this embodiment. The information providing server 3 is installed in, for example, a manufacturer or the like. The information providing server 3 installed in the manufacturer or the like, the communication apparatus 27 of the walking training apparatus 2 installed in a hospital, a facility, or the like, and a communication apparatus 4 installed in a maintenance company are connected through, for example, a communication network 5 such as the Internet, so that they can perform data communication with each other.

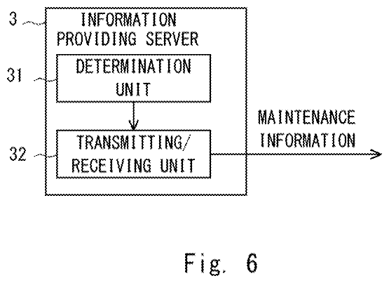

FIG. 6 is a block diagram showing a schematic system configuration of the information providing server according to this embodiment. The information providing server 3 includes a determination unit 31 that determines a timing for performing maintenance for the walking assistance apparatus 23, and a transmitting/receiving unit 32 that transmits/receives data.

The determination unit 31 is a specific example of the determination means. The determination unit 31 compares information about a load and information about a time during which the load is exerted, which have been transmitted from the communication apparatus 27 of the walking training apparatus 2, with a preset evaluation value (i.e., an evaluation value that is set in advance) for the durability of the walking assistance apparatus 23 (hereinafter referred to as the durability evaluation value), and determines a timing for performing maintenance for the walking assistance apparatus 23.

The information about the load is, for example, load values detected by the load sensors 240. The information about the time during which the load is exerted is, for example, the load time during which the load detected by the load sensors 240 is exerted. The timing for performing maintenance for the walking assistance apparatus 23 is, for example, a timing at which sensors such as the load sensors 240 and the angle sensor 239 provided in the walking assistance apparatus 23 are calibrated, or a timing at which consumable components of the walking assistance apparatus 23 are replaced.

The durability evaluation value of the walking assistance apparatus 23 is set in advance in the determination unit 31, and a user can arbitrarily change the set durability evaluation value. The durability evaluation value of the walking assistance apparatus 23 is, for example, a value that is experimentally obtained in advance by carrying out a durability test or the like.

For example, the determination unit 31 calculates an integral value obtained by integrating the load value obtained by each of the load sensors 240 over the load time, and calculates an added value that is obtained by adding up the calculated integral values of these load sensors 240. The determination unit 31 compares the calculated added value with the durability evaluation value. Then, when the added value exceeds the durability evaluation value, the determination unit 31 determines that it is a timing for performing maintenance for the walking assistance apparatus 23 at this stage.

The determination unit 31 may compare, for each of the load sensors 240, the integral value obtained by integrating the load value obtained by that load sensor over the load time with the durability evaluation value, and determine, for each of the load sensors 240 of which the load value exceeds the durability evaluation value, that it is time to perform maintenance for that that load sensor. In this way, it is possible to specify a load sensor(s) 240 that requires maintenance such as calibration.

The determination unit 31 may predict a timing for performing maintenance for the walking assistance apparatus 23 by comparing the integral values obtained by integrating the load values obtained by the load sensors 240 over the load time with the durability evaluation value. For example, the determination unit 31 calculates a difference between the integral values obtained by integrating the load values of the load sensors 240 over the load time with the durability evaluation value. A relation between such differences and elapsed times is experimentally obtained in advance. The determination unit 31 predicts a timing for performing maintenance for the walking assistance apparatus 23 in the future based on the calculated difference and the relation between differences and elapsed times which is obtained in advance.

The transmitting/receiving unit 32 is a specific example of the second transmission means. The transmitting/receiving unit 32 transmits the maintenance information to the communication apparatus 4 or the like of a maintenance company that is registered in advance. The maintenance information is maintenance management recommendation information for recommending that the maintenance and management of the walking assistance apparatus 23 be necessary. The maintenance information includes a maintenance timing for the walking assistance apparatus 23 at this stage and a maintenance timing for the walking assistance apparatus 23 in the future.

The maintenance company performs maintenance such as calibration of sensors of the corresponding walking assistance apparatus 23 and replacement of consumable components according to the maintenance timing received by the communication apparatus 4.

Note that as shown in FIG. 1, the information providing server 3 has, for example, a hardware configuration of a normal computer including a processor 3a such as a CPU (Central Processing Unit) or a GPU (Graphics Processing Unit), an internal memory 3b such as a RAM (Random Access Memory) or a ROM (Read Only Memory), a storage device 3c such as an HDD (Hard Disk Drive) or an SDD (Solid State Drive), an input/output I/F (Interface) 3d for connecting peripheral devices such as a display, and a communication I/F 3e for communicating with an apparatus located outside the information providing server.

By the information providing server 3, it is possible to implement each of the above-described functional components by, for example, having the processor 3a execute a program stored in the storage device 3c, the internal memory 3b, or the like while using the internal memory 3b.

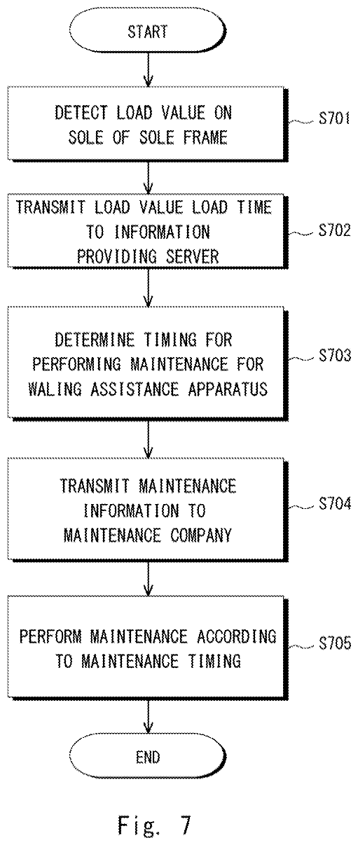

Next, a flow of a maintenance method according to this embodiment will be described. FIG. 7 is a flowchart showing a flow of a maintenance method according to this embodiment.

Each of the load sensors 240 of the walking assistance apparatus 23 of the walking training apparatus 2 detects a load value on the sole of the sole frame 236 and transmits the detected load value to the communication apparatus 27 (step S701).

The communication apparatus 27 transmits the load values obtained by the load sensors 240 and the load time during which these loads have been exerted, which have been received from the walking assistance apparatus 23, to the information providing server 3 (step S702).

The determination unit 31 of the information providing server 3 compares information about the loads and the load time during which the loads have been exerted, which have transmitted from the communication apparatus 27 of the walking training apparatus 2, with the preset durability evaluation value of the walking assistance apparatus 23, and determines a timing for performing maintenance for the walking assistance apparatus 23 (step S703).

The transmitting/receiving unit 32 transmits the timing of the maintenance determined by the determination unit 31 as maintenance information to the communication apparatus 4 of the maintenance company or the like which is registered in advance (step S704).

A person in charge of the maintenance and management in the maintenance company performs maintenance, such as calibration of sensors and/or replacement of consumable components, for the walking assistance apparatus 23 according to the maintenance timing received by the communication apparatus 4 (step S705).

As described above, in this embodiment, the determination unit 31 of the information providing server 3 compares the information about the load and the information about the time during which the load has been exerted, which have been transmitted from the communication apparatus 27 of the walking training apparatus 2, with the preset durability evaluation value of the walking assistance apparatus 23, and determines a timing for performing maintenance for the walking assistance apparatus 23. The transmitting/receiving unit 32 transmits the timing of the maintenance determined by the determination unit 31 to the communication apparatus 4 of the maintenance company or the like as maintenance information.

In this way, the maintenance company or the like can perform maintenance for the walking assistance apparatus 23 at an appropriate maintenance timing by using the maintenance information provided from the information providing server 3.

### Second Embodiment

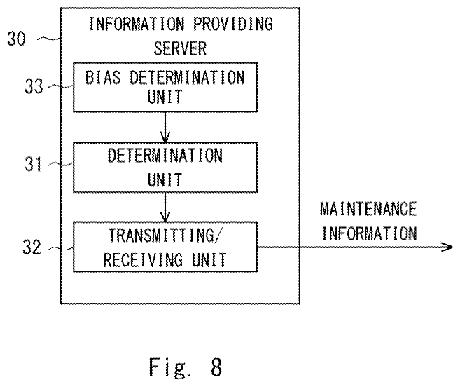

FIG. 8 is a block diagram showing a schematic system configuration of an information providing server according to this embodiment. The information providing server 30 according to this embodiment further includes a bias determination unit 33 that determines a direction of a bias of a load (hereinafter also referred to as a load bias direction) in walking performed by a trainee. The bias determination unit 33 is a specific example of the bias determination means.

The determination unit 31 sets a durability evaluation value according to the load bias direction in the walking of the trainee determined by the bias determination unit 33. As a result, an optimum durability evaluation value is set according to the habit of the trainee because of which the load is biased in his/her walking, thus making it possible to determine a timing for performing maintenance for the walking assistance apparatus 23 more accurately.

The bias determination unit 33 determines the load bias direction in the walking, such as being biased forward, backward, rightward, or leftward, based on information about at least one of the skeletal movement of the trainee, the COP position, and the knee joint angle, which is transmitted from the communication apparatus 27 of the walking training apparatus 2.

For example, when the COP position is closer to the toe, the bias determination unit 33 determines that the load bias direction in the walking is forward based on the COP position transmitted from the communication apparatus 27 of the walking training apparatus 2. Similarly, when the COP position is located on the right side, the bias determination unit 33 determines that the load bias direction in the walking is rightward based on the COP position transmitted from the communication apparatus 27 of the walking training apparatus 2.

The bias determination unit 33 may perform machine learning for data about the skeletal movement and/or the knee joint angle in advance by using a learning machine such as a neural network, and determine the load bias direction in the walking by using the result of the learning.

The durability evaluation value is experimentally obtained by performing a durability test or the like for each of the load bias directions. Durability evaluation values are set while being associated with respective load bias directions.

The determination unit 31 sets a durability evaluation value according to the load bias direction in the walking of the trainee determined by the bias determination unit 33. The determination unit 31 compares the set durability evaluation value with the load values obtained by the load sensors 240 and the load time thereof transmitted from the communication apparatus 27 of the walking training apparatus 2, and determines a timing for performing maintenance for the walking assistance apparatus 23.

For example, the bias determination unit 33 determines that the load bias direction in the walking of the trainee is rightward based on the COP position transmitted from the communication apparatus 27 of the walking training apparatus 2. The determination unit 31 sets a durability evaluation value that is associated with the rightward load determined by the bias determination unit 33. The determination unit 31 compares the durability evaluation value for the rightward load with the load values obtained by the load sensors 240 and the load time thereof transmitted from the communication apparatus 27 of the walking training apparatus 2, and determines a timing for performing maintenance for the walking assistance apparatus 23.

Several embodiments according to the present disclosure have been explained above. However, these embodiments are shown as examples but are not shown to limit the scope of the disclosure. These novel embodiments can be implemented in various forms. Further, their components/structures may be omitted, replaced, or modified without departing from the scope and spirit of the disclosure. These embodiments and their modifications are included in the scope and the spirit of the disclosure, and included in the scope equivalent to the invention specified in the claims.

In the present disclosure, for example, the processes shown in FIG. 7 can be implemented by having a processor execute a computer program.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.).

The program may be provided to a computer using any type of transitory computer readable media. Examples of transitory computer readable media include electric signals, optical signals, and electromagnetic waves. Transitory computer readable media can provide the program to a computer through a wired communication line (e.g. electric wires, and optical fibers) or a wireless communication line.

Note that each of the components constituting the maintenance system 1 according to the above-described embodiments can be implemented not only by a program(s), but also by dedicated hardware such as an ASIC (Application Specific Integrated Circuit) or an FPGA (Field-Programmable Gate Array).

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A maintenance system comprising:
a walking training apparatus comprising:
a walking assistance apparatus comprising a foot attaching part configured to be attached to a foot of a trainee, and load detection means for detecting a load occurring on a sole of the trainee, the load detection means being disposed in the foot attaching part, and the walking assistance apparatus being configured to assist the trainee in walking; and
first transmission means for transmitting information about the load detected by the load detection means and information about a time during which the load is exerted; and
a server comprising:
determination means for comparing the information about the load and the information about the time during which the load is exerted, which have been transmitted from the first transmission means, with a preset evaluation value for durability of the walking assistance apparatus, and determining a timing for performing maintenance for the walking assistance apparatus; and
second transmission means for transmitting the timing of the maintenance determined by the determination means as maintenance information.

2. The maintenance system according to claim 1, wherein
the first transmission means transmits the load detected by the load detection means and the load time during which the load is exerted to the server, and
the determination means of the server calculates an integral value obtained by integrating the load transmitted from the first transmission means over the load time, compares the calculated integral value with the evaluation value for the durability, and determines the timing for performing the maintenance for the walking assistance apparatus.

3. The maintenance system according to claim 1, wherein
the first transmission means transmits, to the server, information about at least one of a skeletal movement of the trainee, a center of a load exerted on the sole, and a knee joint angle detected by a sensor provided in the walking training apparatus together with the information about the load and the information about the time during which the load is exerted,
the server further comprises bias determination means for determining a direction of a bias of the load in the walking of the trainee based on the information about at least one of the skeletal movement of the trainee, the center of the load exerted on the sole, and the knee joint angle transmitted from the first transmission means,
the evaluation value for the durability is set according to the direction of the bias of the load, and
the determination means sets the evaluation value for the durability according to the direction of the bias of the load in the walking of the trainee determined by the bias determination means, compares the set evaluation value for the durability with the information about the load and the information about the time during which the load is exerted, which have been transmitted from the first transmission means, and determines the timing for performing the maintenance for the walking assistance apparatus.

4. A maintenance method comprising:
detecting a load occurring on a sole of a trainee to which a walking assistance apparatus is attached, and the walking assistance apparatus being configured to assist the trainee in walking;
transmitting, to a server, information about the detected load and information about a time during which the load is exerted;
comparing, by the server, the transmitted information about the load and the information about the time during the load is exerted with a preset evaluation value for the durability of the walking assistance apparatus, and determining a timing for performing maintenance for the walking assistance apparatus; and
transmitting, by the server, the determined timing of the maintenance as maintenance information.

5. A non-transitory computer readable medium storing a program for causing a computer to perform:
a process for detecting a load occurring on a sole of a trainee to which a walking assistance apparatus is attached, and the walking assistance apparatus being configured to assist the trainee in walking;
a process for transmitting, to a server, information about the detected load and information about a time during which the load is exerted;
a process for comparing, by the server, the transmitted information about the load and the information about the time during the load is exerted with a preset evaluation value for the durability of the walking assistance apparatus, and determining a timing for performing maintenance for the walking assistance apparatus; and
a process for transmitting, by the server, the determined timing of the maintenance as maintenance information.

6. A maintenance system comprising:
a walking training apparatus comprising:
a walking assistance apparatus comprising a foot attaching part configured to be attached to a foot of a trainee, and a load detector configured to detect a load occurring on a sole of the trainee, the load detector being disposed in the foot attaching part, and the walking assistance apparatus being configured to assist the trainee in walking; and
a first transmitter configured to transmit information about the load detected by the load detector and information about a time during which the load is exerted; and
a server comprising:
a determination unit configured to compare the information about the load and the information about the time during which the load is exerted, which have been transmitted from the first transmitter, with a preset evaluation value for durability of the walking assistance apparatus, and determining a timing for performing maintenance for the walking assistance apparatus; and
a second transmitter configured to transmit the timing of the maintenance determined by the determination unit as maintenance information.
